(19) United States
(12) Patent Application Publication
(10) Pub. No.: US 2009/0326449 A1
(43) Pub. Date: Dec. 31, 2009

(54) Control apparatus of catheter feeder

(75) Inventors: Jaw-Lin Wang, Taipei (TW); Been-Der Yang, Taipei (TW); Chi-Lin Yang, Taipei (TW); Wei-Cheng Huang, Taipei (TW); Yao-Hung Wang, Taipei (TW); Tzu-Cheng Lin, Taipei (TW)

Correspondence Address: BACON & THOMAS, PLLC, 625 SLATERS LANE, FOURTH FLOOR, ALEXANDRIA, VA, 22314-1176, US

(73) Assignee: National Taiwan University, Taipei (TW)

(21) Appl. No.: 12/285,814

(22) Filed: Oct. 15, 2008

(30) Foreign Application Priority Data
Jun. 25, 2008 (TW) 097123638

(51) Int. Cl. A61M 25/092

(52) U.S. Cl. 604/95.01

(57) ABSTRACT

The invention is disclosed to a control apparatus of catheter feeder. It comprises two rotatable mechanisms and a transmittal device that can propel the guidewire into continuous motion comprising moving forward, moving backward, moving clockwise, and moving counter-clockwise. The invention comprises of a square frame and three gears that are meshed together and are referred to the first, second, and third gear, respectively. At the front of the third gear are the first and second sets of the idle wheels that can hold the guidewire tightly. The invention employs the mechanical gears to control movement of the guidewire.

### BACKGROUND OF THE INVENTION

1. Field of the Invention

The invention is disclosed to a control apparatus of catheter feeder, and more particularly, to a mechanical gear-type control apparatus of catheter feeder.

2. Description of the Prior Art

At present the method used to diagnose the cardiovascular disease is the coronary angiography, which is commonly called the cardiac catheterization. The cardiac catheterization is the medical angioplasty for examining or curing various heart diseases by the invasive catheter, which can be used to take the photograph for coronary artery, aortic and pulmonary, and can be used to measure the pressure of heart at every position as well.

When the cardiac catheterization is carried out, local anesthesia has to be conducted at first. Then a small wound is cut smaller than 0.5 cm on the skin. Stretch the catheter into the coronary artery through the peripheral aortic of thigh or arm. Then inject the developer, employ the X-ray instrument to take the photo for the flow state of blood in coronary artery, in order to understand the precise position and order of severity for the cardiovascular disease.

Through preliminary estimation, about ⅓ of patients having cardiovascular disease is suitable to be treated by percutaneous coronary intervention of internal medicine, which is called the catheter operation commonly. The method used to treat the obstruction of coronary artery, such as the percutaneous transluminal coronary angioplasty, coronary in-stent restenosis etc., also can be conducted by the catheter operation.

As for the catheter equipment, the doctor will cut a small wound on the femoral artery of thigh or the radial artery of arm at first, and put the sleeve of catheter equipment into as the entrance. Then put into the guidewire through the sleeve of catheter equipment, and guide the catheter equipment by the guidewire to the disease position. The guidance process of catheter equipment is very complicated, because the blood vessel of human is very zigzag with small specific elasticity. Thus, when the guidewire is moved in human body, it should be very cautious to prevent any unexpected accident of operation. Therefore, the guidance and control of catheter is extremely important.

Through searching the patent literatures in USA patent database, U.S. Pat. No. 4,920,980 “Catheter with controllable tip”, U.S. Pat. No. 5,055,109 “Torque transmitting assembly for intravascular devices”, U.S. Pat. No. 5,178,158 “Convertible guidewire-catheter with soft tip”, U.S. Pat. No. 5,190,050 “Tip deflectable steerable catheter”, U.S. Pat. No. 5,489,270 “Controlled flexible catheter”, U.S. Pat. No. 5,957,941 “Catheter apparatus and drive assembly thereof”, U.S. Pat. No. 6,706,018B2 “Adjustable length catheter assembly”, and U.S. Pat. No. 7,267,663B2 “Apparatus for guide catheter positioning” have shown the traditional control device or control method of catheter feeder. The push and pull way of socket-type and manual operation are nearly adopted as their design method, which do not totally match with the actual demand and application of guidewire control.

U.S. Pat. No. 4,920,980 “Catheter with controllable tip”, U.S. Pat. No. 5,178,158 “Convertible guidewire-catheter with soft tip”, U.S. Pat. No. 5,190,050 “Tip deflectable steerable catheter”, U.S. Pat. No. 5,489,270 “Controlled flexible catheter”, and U.S. Pat. No. 6,706,018B2 “Adjustable length catheter assembly” described the control equipment of guideware tip for free moving. The manual operation is applied, which does not match with the habitual operation method of doctor and does not have actual application function.

U.S. Pat. No. 5,957,941 “Catheter apparatus and drive assembly thereof” described an automatic control apparatus. A rotatable mechanism is installed on a linear mechanism. The rotatable mechanism controls the rotation of guidewire, and the linear mechanism controls the advancement of guidewire. However, its advancing stroke is limited, and the location of motor is not good enough, the whole mechanism is very complicated, which lacks the practicability.

Thus, in order to operate and control the catheter feeder more efficiently, it is necessary to develop a control apparatus, which can control the movement of guidewire easily in the human body, to improve the guidance efficiency of catheter feeder and reduce the transport time of catheter in the human body.

### SUMMARY OF THE INVENTION

The invention is disclosed to an automatic control apparatus of catheter feeder. There are two rotatable mechanisms and a transmittal device that can propel the guidewire processing continuous motion comprising moving forward, moving backward, moving clockwise, and moving counter-clockwise. The invention comprises of a square frame and three gears that are meshed together and are referred to the first, second, and third gear, respectively. The first and second sets of the idle wheels are installed at the front of the third gear that can hold the guidewire tightly.

The feature of the invention is to employ the mechanical gear to transfer the holding things for controlling the movement of the guidewire.

The advantages of the invention are low cost, simple and easy apparatus, and high mobility etc., which has big design novelty and industrial utilization.

The advantage of the invention is a simple device which can be carried conveniently. It is not necessary to use the complicated driven equipment, which can be applied in the medical field easily.

The advantage and spirit of the invention can be understood further by the following detail description of invention and attached Figures.

### BRIEF DESCRIPTION OF THE DRAWINGS

FIG. 1A and 1B show the control principle of the invention.

FIG. 2 shows the side view of the invention.

FIG. 3 shows the perspective drawing of the invention.

FIG. 4 shows the exploded drawing of the invention.

FIG. 5 shows the embodiment drawing of the invention.

### DESCRIPTION OF THE PREFERRED EMBODIMENT

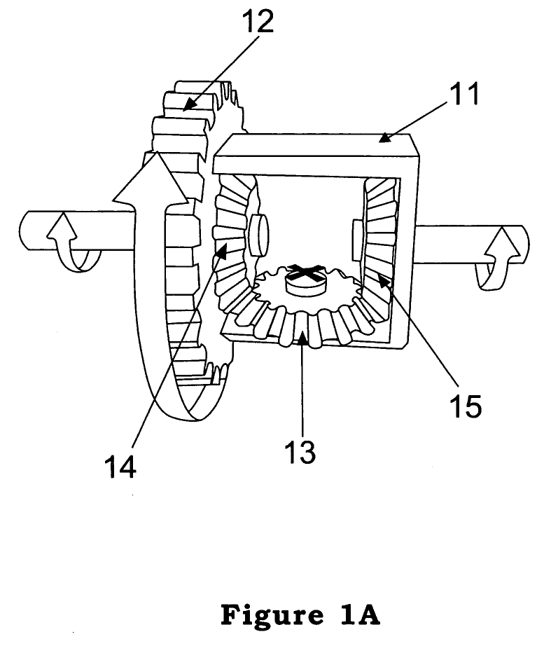

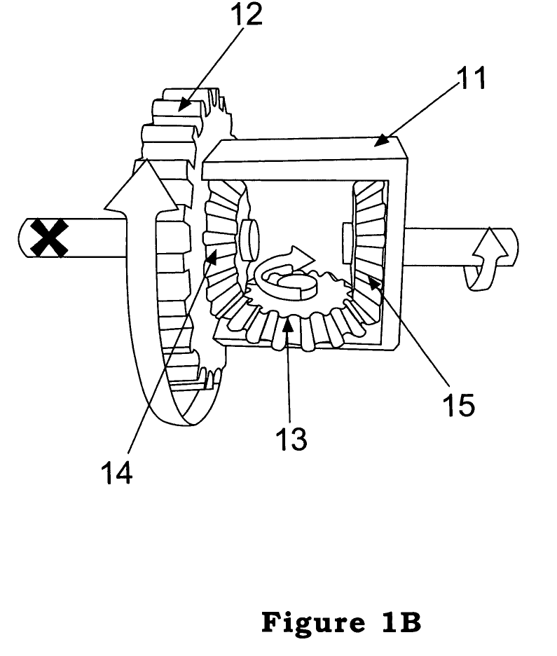

The invention is disclosed to a control apparatus of catheter feeder. Its basic control principle is shown in FIG. 1A and 1B. Its operation principle is similar to the differential mechanism of vehicle. FIG. 1A shows forward motion and FIG. 1B shows turning motion.

In FIG. 1A, the big gear 12 is connected to the square frame 11. After receiving the power from the motor, it will drive the middle gear 13 to rotate forward together, and the left gear 14 and right gear 15 will also rotate forward simultaneously are under the leading of middle gear 13, to produce the forward motion.

In FIG. 1B, when turning is carried out, the left gear 14 will be blocked. Because the left gear 14 is unable to move, the middle gear 13 driven by the big gear 12 will carry on rotation. The right gear 15 is driven to accelerate the rotational speed to turn, then turning motion is carried out.

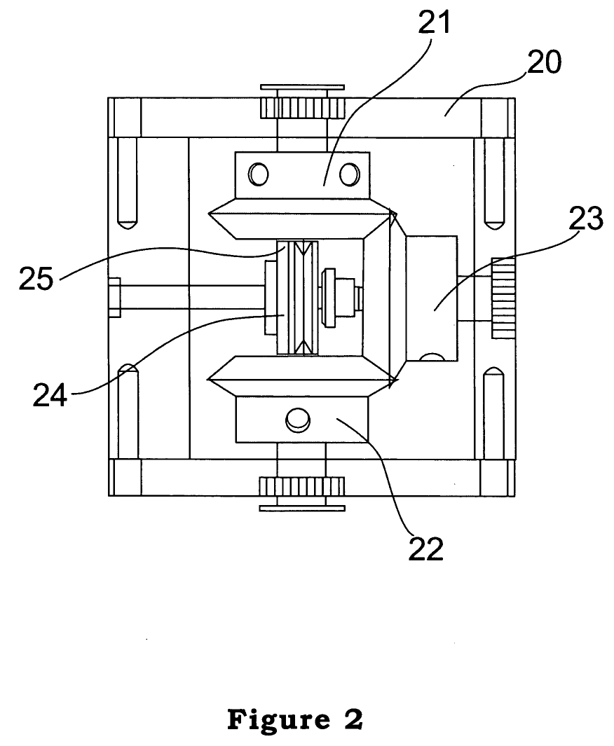

FIG. 2 shows the side view of the control apparatus of catheter feeder of the invention. It comprises a frame 20 to be load and fix every component. The first gear 21 comprises a first hollow axis for the catheter and guidewire passing through. The second gear 22 comprises a second hollow axis for the catheter and guidewire passing through. The first gear 21 and the second gear 22 are meshed in parallel. The third gear 23 is meshed to the first gear 21 and the second gear 22 to transmit the power. There is the first idle wheel set 24 at the front of the third gear 23, and the second idle wheel set 25 installed in frame 20 that can hold the guidewire tightly. The outer circle ring for the first idle wheel set 24 and the second idle wheel set 25 can be surrounded by the elastic material having high friction, such as the rubber materials.

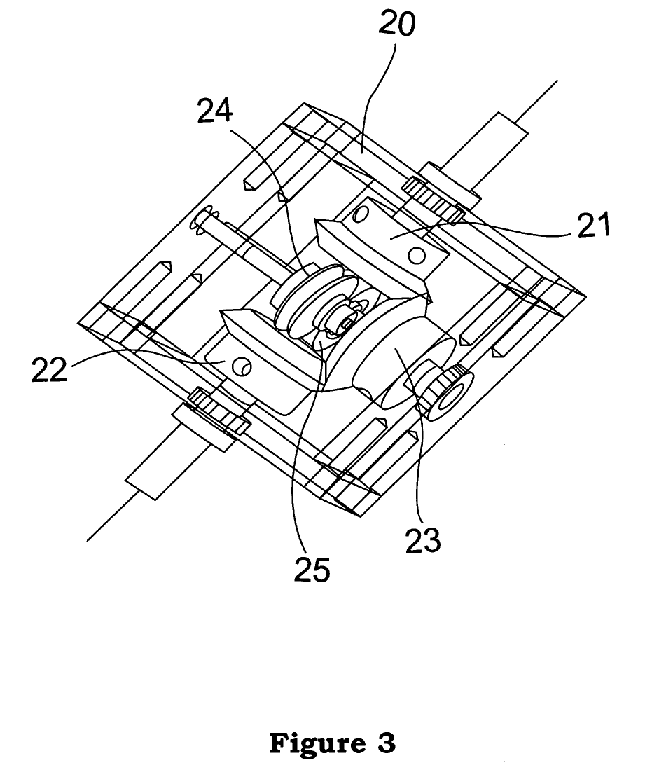

FIG. 3 shows the perspective drawing of the invention. There is the first idle wheel set 24 at the front of the third gear 23 and the second idle wheel set 25 on the other side, both of the first idle wheel set 24 and the second idle wheel set 25 can hold the guidewire tightly.

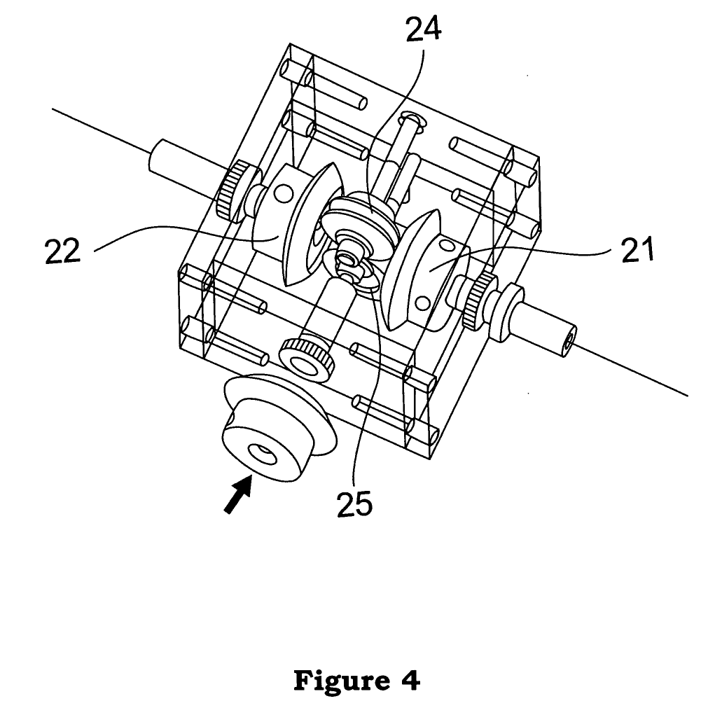

FIG. 4 shows the exploded drawing of the invention. The first idle wheel set 24 and the second idle wheel set 25 are installed in parallel for holding the guidewire tightly.

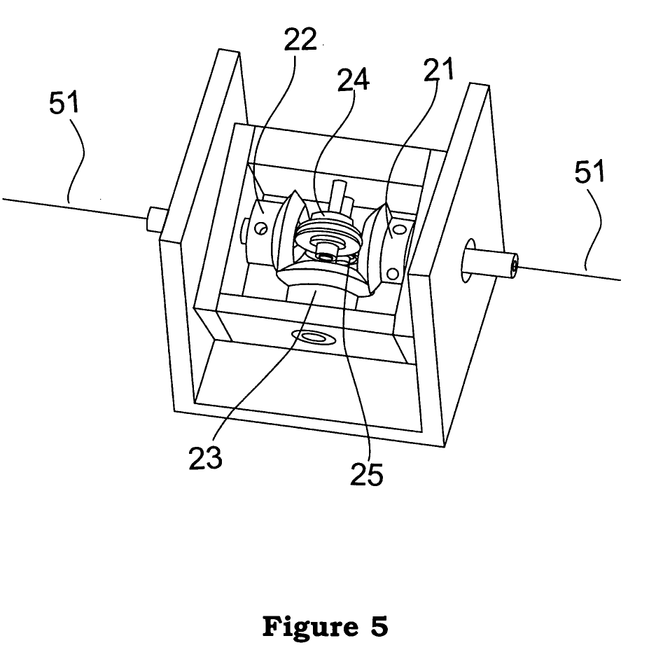

As FIG. 5 shows that the embodiment drawing of the invention. There is a guidewire 51 that passes through the first gear 21 and the second gear 22. When the motor inputs power to the first gear 21 and the second gear 22, the first gear 21 and the second gear 22 can drive rotation of the third gear 23. When the first gear 21 and the second gear 22 rotate a in the same direction and at the constant speed, the first gear 21 and the second gear 22 will force the third gear 23 to rotate around the central axis of the first gear 21 and the second gear 22, which also drive rotation of the square frame 20, the first idle wheel set 24 and the second idle wheel set 25 at the same time. In the meanwhile, the first idle wheel set 24 and the second idle wheel set 25 will hold the guidewire 51 tightly. The guidewire 51 will rotate accordingly to become a rotatory motion.

In addition, when the power inputted to the first gear 21 and the second gear 22 is in the reverse direction and at the constant speed, the third gear 23 will rotate itself. The third gear 23 will drive the first idle wheel set 24, and then the first idle wheel set 24 and the second idle wheel set 25 will hold the guidewire 51 tightly. The guidewire 51 will be pushed out accordingly to become a forward motion.

The invention is disclosed to a control apparatus of catheter feeder. It mainly comprises two rotatable mechanisms and a transmittal device that can propel the guidewire into continuous motion comprising moving forward, moving backward, moving clockwise, and moving counter-clockwise.

The feature of the invention is to employ the mechanical gears to control movement of the guidewire. The advantages of the invention are low cost, simple and easy apparatus, and high mobility etc., which is big design novelty. The other advantage of the invention is a simple device which can be carried conveniently. It is not necessary to use the complicated driven equipment, which can be able to bear the high temperature sterilization environment and can be applied in the medical field widely.

It is understood that various other modifications will be apparent to and can be readily made by those skilled in the art without departing from the scope and spirit of this invention. Accordingly, it is not intended that the scope of the claims appended hereto be limited to the description as set forth herein, but rather that the claims be construed as encompassing all the features of patentable novelty that reside in the present invention, including all features that would be treated as equivalents thereof by those skilled in the art to which this invention pertains.

What is claimed is:

1. A control apparatus of catheter feeder, comprising:
two rotatable mechanisms, said two rotatable mechanisms controlling a transmittal apparatus, said transmittal apparatus propelling a guidewire to process continuous moving forward, moving backward, moving clockwise, and moving counter-clockwise, to form said control apparatus of catheter feeder.

2. The control apparatus of catheter feeder according to claim 1, wherein two rotatable mechanisms comprise moving clockwise and moving counter-clockwise, said moving clockwise and said moving counter-clockwise is driven by a motor.

3. The control apparatus of catheter feeder according to claim 1, wherein said transmittal device comprises a plurality of idle wheel sets, said plurality of idle wheel sets control a guidewire tightly by transmitting holding things.

4. A control apparatus of catheter feeder, comprising:
a square frame, said square frame loading a plurality of components;
a plurality of gears, said a plurality of gears being meshed together to transmit a power through a catheter and a guidewire; and,
a plurality of idle wheel sets, said plurality of idle wheel sets installed near said plurality of gears for holding said catheter tightly and said guidewire to form said control apparatus of catheter feeder.

5. The control apparatus of catheter feeder according to claim 4, wherein said plurality of gears comprises 3 gears.

6. The control apparatus of catheter feeder according to claim 4, wherein said plurality of idle wheel sets is driven by a motor.

7. The control apparatus of catheter feeder according to claim 4, wherein the plurality of idle wheel sets comprises 2 idle wheel sets.

8. A control apparatus of catheter feeder of catheter feeder, comprising:
a square frame, said square frame been loading a plurality of components;
a first gear, said first gear having a first hollow axis for passing through a catheter and a guidewire;
a second gear, said second gear having a second hollow axis for passing through said catheter and said guidewire;
a third gear, said third gear been meshed to said first gear and said second gear to transmit a power; and
a plurality of idle wheel sets, said plurality of idle wheel sets meshed at a front of said third gear for holding said catheter and said guidewire tightly to form the control apparatus of catheter feeder.

9. The control apparatus of catheter feeder according to claim 8, wherein said first gear and said second gear are meshed in parallel.

10. The control apparatus of catheter feeder according to claim 8, wherein said first gear and said second gear are driven by the motor.

11. The control apparatus of catheter feeder according to claim 8, wherein the outer circle ring surrounded the idle wheel set comprises an elastic material having high friction.

12. The control apparatus of catheter feeder according to claim 11, wherein elastic material having high friction comprises the rubber.
